(19) United States
(12) Patent Application Publication
(10) Pub. No.: US 2016/0089379 A1
(43) Pub. Date: Mar. 31, 2016

(54) TREATMENT OF NEUROLOGICAL AND OTHER DISORDERS

(71) Applicants: Alexander George Brian O'NEIL, Subiaco, Western Australia (AU); PALMAYA PTY LTD, Subiaco, Western Australia (AU)

(72) Inventor: Alexander George Brian O'Neil, Subiaco, WA (AU)

(21) Appl. No.: 14/891,836

(22) PCT Filed: May 20, 2014
(86) PCT No.: PCT/AU2014/000527

§ 371 Date: Nov. 17, 2015

(30) Foreign Application Priority Data
May 20, 2013 (AU) 2013901789

(51) Int. Cl. A61K 31/5517; A61K 31/485; A61K 31/5513

(52) U.S. Cl. CPC A61K 31/5517; A61K 31/5513; A61K 31/485

(57) ABSTRACT

Methods and implants for treating neurological, muscular and other cells having an electrical potential, with an infusion of antagonists or inverse agonists (such as flumazenil for the benzodiazepine receptor, or naltrexone for the opiate receptor or other antagonist for other receptor) at rates that are so low that only a small percentage of receptors have the antagonist effect at any one time.

### TECHNICAL FIELD

The present invention relates to methods and implants for treating neurological, muscular and other cells having an electrical potential, with an infusion of antagonists or inverse agonists (such as flumazenil for the benzodiazepine receptor, or naltrexone for the opiate receptor or other antagonist for other receptor) at rates that are so low that only a small percentage of receptors have the antagonist or inverse agonist effect at any one time.

### BACKGROUND ART

The following discussion of the background art is intended to facilitate an understanding of the present invention only. The discussion is not an acknowledgement or admission that any of the material referred to is or was part of the common general knowledge as at the priority date of the application.

Endogenous or synthetic compounds that interact with receptors can be classified as agonists, inverse agonists, or antagonists. An agonist is a compound that is able to elicit a response following receptor occupation and activation. An inverse agonist is a compound that binds to the same receptor binding-site as an agonist but reverses the activity of receptors, thereby exerting the opposite effect of a receptor agonist. Antagonists are compounds that bind to the receptor in a reversible way without activating the effector system for that receptor.

Neurotransmitters are endogenous chemicals that transmit a signal from the neuron to its target cell. The main neurotransmitter controlling excitement state in the brain is GABA (γ-aminobutyric acid). For normal brain function, the overall balance between neuronal excitation and inhibition must be maintained. If the balance is upset, a range of conditions may result due to lack of inhibition/excitation or too much inhibition/excitation at the receptor site. In diseased states and with age, the balance between benzodiazepine agonists and inverse agonists or other antagonists shifts towards the benzodiazepine agonists.

GABA acts on a variety of GABA receptors in the brain. There are a large number of different GABA receptors, including GABA-A receptors. In addition to the primary binding sites for GABA, the GABA-A receptor has other secondary binding sites for molecules that modulate the effect of GABA, such as benzodiazepines, barbiturates, steroids, and alcohol.

The general understanding is that these modulating agents alter the efficiency of chloride ion transfer into the cell. This change modifies the size of the channel, which in turn modifies the receiving neuron's permeability to chloride ions. Since chloride ions are negatively charged, when they enter the neuron, they hyperpolarize the cell.

As mentioned above, it is believed that the class of drugs known as the benzodiazepines interacts at the surface of the cell at the GABA receptor. When GABA interacts with its receptor site, the result is a trickle of ions into the cell. However, when a benzodiazepine interacts with the GABA receptor, it amplifies the GABA effect of ions into the cell, resulting in hyperpolarisation of the cell, and cell damage or cell death.

Current examples of disease treatments mostly depend on the use of agonists. In the case of hypersomnia, stimulants are used most commonly, such as dexamphetamine, methylphenidate and modafinil. Experiments with a single, high dose injection of flumazenil have shown promise but have been associated with side effects, such as fitting and anxiety.

In treating Parkinson's disease, treatment has relied on the use of dopamine replacement, rather than allowing valium to make the cells produce their own dopamine.

The present invention seeks to overcome, or at least ameliorate, one or more of the deficiencies of the prior art mentioned above, or to provide the consumer with a useful or commercial choice.

### SUMMARY OF INVENTION

In one aspect of the present invention there is provided a method for treating a neurological disease, or other disease or disorder associated with damage to cells having an electrical potential, comprising administering a continuous infusion of a benzodiazepine antagonist or inverse antagonist at a rate of 0.001 micrograms to 20,000 micrograms per hour to a patient in need thereof.

In a preferred embodiment, the continuous infusion of the benzodiazepine antagonist is maintained for a period of time of more than 4 days, at least 10 days, at least 20 days, at least 30 days, at least 40 days, at least 50 days, at least 60 days, at least 70 days, at least 80 days, at least 90 days, at least 100 days, at least 150 days, at least 200 days, at least 250 days, at least 300 days or more. In another preferred embodiment the continuous infusion of the benzodiazepine antagonist is maintained indefinitely.

In another aspect of the present invention there is provided an implant comprising a benzodiazepine antagonist. The implant comprises at least a benzodiazepine antagonist or inverse antagonist in an amount sufficient for the implant to release the benzodiazepine antagonist at a continuous rate of 0.001 micrograms to 20,000 micrograms per hour for a prolonged period of time.

In one highly preferred embodiment, the antagonist is flumazenil.

In one embodiment, the implant releases the benzodiazepine antagonist at a rate of between 10 to 300 micrograms per hour. In a further embodiment, the benzodiazepine antagonist at a rate of between 300 to 500 micrograms per hour. In a further embodiment, the benzodiazepine antagonist is released at between 500 to 1,000 micrograms per hour. Further still, the release rate of the benzodiazepine antagonist from the implant may be at a rate of up to 20,000 micrograms per hour.

In another aspect of the present invention there is provided a method for treating a neurological disorder, disease or condition comprising administering a continuous infusion of the antagonist flumazenil or an inverse agonist as well as the benzodiazepine agonist valium or alternative benzodiazepine agonist.

In a preferred embodiment, the benzodiazepine antagonist or inverse agonist is valium or a valium-like substance.

In a highly preferred embodiment for the treatment of diseases, the benzodiazepine antagonists and agonists may be required while in many circumstances a slow deliver of antagonists only is required.

In a further aspect of the present invention there is provided an implant comprising flumazenil and the L-isomer of naltrexone. In another embodiment, the implant comprises flumazenil and the R-isomer of naltrexone.

In a further aspect of the invention there is provided the use of an implant to treat a neurological disease, or other disease or disorder associated with cells having an electrical potential in a patient, wherein the implant comprises a benzodiazepine antagonist, wherein the implant comprises at least a benzodiazepine antagonist or inverse antagonist in an amount sufficient for the implant to release the benzodiazepine antagonist at a continuous rate of 0.001 micrograms to 20,000 micrograms per hour for a prolonged period of time.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a graphical representation of the percentage of flumazenil released from implants produced with poly-lactic acid membranes.

FIG. 2 is a graphical representation of the tapping speed test from a patient suffering from Parkinson's Disease before and after administration with flumazenil.

### DESCRIPTION OF EMBODIMENTS

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, displacement and field strength). A range of values will be understood to include all values within the range, including the values defining the range, and values adjacent to the range which lead to the same or substantially the same outcome as the values immediately adjacent to that value which defines the boundary to the range.

The term neurological “disorder”, disease”, or “condition” may be used interchangeably to define a disease, disorder or condition which effects the body's nervous system.

The term “treatment” as used herein covers any treatment of a disease in an animal (including a human), and includes: (i) preventing the disease from occurring; (ii) inhibiting the disease, i.e., arresting its development; (iii) relieving the disease, i.e., causing regression of the disease; or (iv) modifying normal biological activity.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

Throughout this specification, unless the context requires otherwise, the word “comprise” or variations such as “comprises” or “comprising”, will be understood to imply the inclusion of a stated integer or group of integers but not the exclusion of any other integer or group of integers.

### DETAILED DESCRIPTION OF THE INVENTION

The applicant has surprisingly found that a continual infusion of a low dose of antagonist or inverse agonist, such as flumazenil to the benzodiazepine GABA receptor, or naltrexone to the opiate receptor, up-regulates receptors and cell function that drive neurological disease patterns, as well as correcting cell damage caused from hyperpolarisation and inactivity of the cell.

Neurological disorders of the brain and organs (for example, the endocrine systems, islet cells of the pancreas, the eyes, hearing and so forth) depend on the neural function operating on an electrical system that requires constant changes in the voltage in cells.

We have discovered a way of making brain cells recover their function after they have become non-functional and unable to conduct a current on a long term basis and so allow the recovery of these cells from the effects of diseases and ageing. This discovery allows the recovery of function for individual neurons that have literally been “switched off” by their disease process or ageing over long periods of time. In this switched off state, they are close to cell death. This contributes to the progress of neurological diseases. In addition hair cells in the cochlear may show recovery. In the case of muscle cells, the infusion may protect myocardial cells death post infarct due to the change in the electrical system.

The prior art does not reflect an understanding that in the case of neurological disorders in general, this correction could return cells to function on a long term basis if these cells were not dead, simply by delivering a treatment associated with the delivery of a molecule to each cell on a repeated basis within an operating time in between where the cell is returned to the normal operating range before the next molecule was returned to correct the operating voltage again.

This corrective process has been achieved for neurons in the nervous system by the present invention, which is to deliver molecules, such as benzodiazepine antagonists or inverse agonists on a continuous basis through an implant that provides a continuous drug delivery system for many months at a time (preferably more than 6 months) or for the life span of the patient, which releases the molecules that repair the operating voltage to the operating level that will receive current from the rest of the central nervous system. The implants in their ideal design should be biodegradable and easily replaced 1-3 times per year or less so that the intermittent repair of every neuron occurs on a regular basis.

The corrective process is usually only required in disease states, but the principal can also be applied to ageing and so prolong survival of the organism, as correction on a regular basis of the cell voltage corrects neuronal dysfunction even in autoimmune disease and also may alter the course of some malignancies electrical activity in cells as the central nervous system may play a corrective function in reasons for these diseases.

The cell saving mechanism of the treatment of the present invention is to return the GABA benzodiazepine receptor sensitivity to its original level so that valium or valium-like substances are able to act at its receptor site. The entry of the benzodiazepine antagonist or inverse agonist has a cell saving effect. That is the correction of the benzodiazepine receptor by the antagonist molecule which allows the recognition of the next valium or benzodiazepine molecule which causes the release of dopamine and restarting of the inactive cell where moving its voltage back to the operating current range becomes possible. This is demonstrated in the functional benefits in patients with neurological conditions where we have seen massive patient benefits.

The invention and the research associated with the invention has confirmed the applicant's belief that flumazenil delivered at a rate of between about 0.001 micrograms per hour to about 20,000 micrograms per hour, and preferably 10 to 300 micrograms per hour, allows valium or valium-like substances to enter a number of central nervous system cells in a manner that would exclude those cells from functioning for a moment in time.

At the time of filing, the most relevant prior art held by the same applicant provided an understanding that receptor function could be corrected by benzodiazepine antagonists and inverse agonists but an understanding that this would have to be continued on a long term recycling basis, possibly for life to achieve sustained recovery of the cell did not exist. Nor did this understanding extend to the fact that this could be applied to all neurological diseases, muscle tissue diseases or autoimmune disease.

The current invention is distinguished from the prior art as it makes available a long term sustainable solution to bring symptomatic relief and arrest neurological disorders by correcting the allosteric relationship of the GABA receptor and the benzodiazepine receptor again and again, and on a regular basis so that the cell function is continually protected by being able to be constantly returned regularly to normal operating voltage. This prevents the cell damage or cell death.

Neurological diseases may be “sparked off” by ischemic insult, viral insult, autoimmune disorders, chemical insult, trauma or other injury, and the central nervous system's response is to release substances which have benzodiazepine activity. Providing the injury is of short duration this is protective to the cell and the arrival of valium or valium-like substances facilitates the protection of the cell by taking it out of the operating voltage (i.e. rest from currents) to a higher voltage for a period of time to allow cell recovery.

Our experiments have proved that we can return these cells to normal function. That is, we have demonstrated this in patients with abnormal neurone function, including hypersomnia, epilepsy, and Parkinson's disease, and we expect this will also follow for diseases such as depression, bi-polar, mania, Attention Deficit Disorder (ADD), Attention Deficit Hyperactivity Disorder (ADHD), psychosis, and other sleep disorders, decreased cognitive function with age or disease, stroke, and spinal cord injury.

We can do this in a sustained manner with a long term implant which is replaced several times a year to deliver a regular cycle of benzodiazepine antagonists or inverse agonists at a rate that regularly corrects receptor sensitivity.

Most, if not all scientists and research groups have assumed that the number of live cells are reduced, whereas the real problem identified by the applicant is that the benzodiazepine active molecules have interfered with, masked or destroyed the allosteric linkages at the cells in the diseased area and the number of cells which are in a state of hyperpolarisation are not conducting a normal or effective current. If they stay in this state long enough, they die.

This invention contrasts with this work of others where intravenous or oral doses of flumazenil at rates of flumazenil as high as 20,000 micrograms were administered as bolus doses. In doses as high as 20,000 micrograms, patients have anxiety attacks or fits if benzodiazepine molecules are striped from the receptors suddenly. Recent experiments by the applicant have confirmed that sustained long term regular delivery of antagonists or inverse antagonists result in recovery of the receptor, and treatment of neurological disease states.

The applicant concludes from the experiments disclosed herein that this corrective process can be applied to all neurological disorders, including autoimmune disorders or disorder previously considered to result from the autoimmune system, as well as damaged muscle tissues associated with cardiovascular disease.

Loss of cell function resulting from hyperpolarisation of the cell cannot be corrected until endogenous benzodiazepines (naturally occurring molecules that bind to the same site as synthetic benzodiazepines) can re-enter or influence the cell. This can only occur after fixing the receptor. With the hyperpolarised cell, transmission of electrical signals stops. It is suggested by the applicant that illness and loss of cell function gives the appearance of cell death. In addition, neuroscientists have assumed that they can count the number of functional receptor sites left following cell death by measuring the binding of radioactive molecules of drugs that would normally be taken up by these sites. That is, these studies show that the benzodiazepine receptor sites appear to be reduced in illness, concluding the cells have died. The applicant believes that in actual fact, the endogenous benzodiazepines are blocking these sites or alternatively the damage to the benzodiazepine receptor site is such that it is more difficult for the flumazenil to bind. Thus, the results generated from these radio-active uptake studies incorrectly report cell damage or cell death.

Thus, applicant has identified for the first time the mechanism underlying neural cell damage or cell death after an injury is the hyperpolarisation of the neural cell, resulting from an excess of valium or valium-like substances. The excess valium or valium-like substances causes hyperpolarisation of the cell, leading to cell damage and eventually cell death. A hyperpolarised cell is unable to respond to molecules, such as valium until the cell benzodiazepine receptor site recovers from the uncoupling of the allosteric linkage of the GABA and benzodiazepine sites caused by prolonged exposure of valium or benzodiazepine active molecules at the benzodiazepine receptor site.

### Screen Saver Analogy

The applicant has likened this cell saving, or brain saving process to a screen saver on a computer. With a computer monitor screen, the picture is made up of thousands of pixels. The larger the screen the more pixels and each pixel has the ability to be in a variety of states. It can show a single colour (such as white, blue, red or green) or it can be turned off (e.g. when the computer is turned off). When a computer is used for normal processes such as running different programs, the pixels are rapidly cycling through these states for the monitor screen to always display what the computer is programmed to show.

However, in some circumstances the computer monitor is left on for a long period of time, with the screen showing an image. While the screen is displaying the same image each and every pixel is unable to change—it is constantly sent the same message by the computer to show each pixel in the same frequency and voltage. After an extended period of time the screen image is damaged, and is generally referred to as “screen burn”. Even when different programs are in use on the computer, there will still be an outline of the image that was on the screen when the “screen burn” occurred. Screen burn is like a constant ghosting caused by a percentage of pixels that are permanently “stuck” in their old state.

The solution to reversing this “screen burn” (and also a method of prevention) is known as a “screen saver”. What a “screen saver” does is to slowly update and change each and every pixel on the screen. It ensures that no single pixel on that screen stays in the same state for too long—and hence prevents any chance of “screen burn” from the same image being displayed for too long. But over time it can also reverse “screen burn” in some cases as by regularly sending different messages to a “burnt” pixel so that it might eventually start to respond again.

Thus, applicant believes a similar situation occurs with neurons in the brain. The billions of neurons in the brain are constantly receiving electrical signals. But sometimes diseases or environmental factors can send the same signal to a set of neurons constantly for so long that the neurons become hyperpolarised (the equivalent of “screen burn” for the brain). When neurons are suffering from hyperpolarisation they do not function as they are supposed to and this manifests as symptoms for the patient such as fatigue, loss of cognition, poor impulse control as some of the neurons that would control impulses are not carrying currents. What the low flumazenil infusion does is deliver a molecule of flumazenil to an un-coupled damaged benzodiazepine receptor site.

When the flumazenil molecule leaves the site the re-coupling following the exit of the molecule will allow, for example valium to start the cell working with dopamine releases and full cell function which restores this cell to the operating range of the current taking information from one cell to another. It is for this reason the patients have full vigilance in hypersomnia patients when they have not experience it before, as well as dopamine release from their own cells in Parkinson's disease and cease peti mal fits (staring spells) with our epilepsy patient.

Without being bound by theory, the applicant believes that low but constant doses of antagonists or inverse agonists, like flumazenil, are effective because they constantly cycle the neurons through different states—in a similar way to a screen saver with the pixels on a monitor screen. And while it is possible that some neurons have been hyperpolarised for so long they are beyond recovery, it seems that this approach can restore many hyperpolarised neurons back to a point where they once again respond to their ‘switch’ that sets their state. The hyperpolarised neurons can be returned to a ‘healthy’ state where they not only recover from the “screen burn” but they also have their switch repaired so that in the future they respond well to electrical signals.

Some of the processes of cell recovery are assisted by correcting the function of a number of neurons by allowing the retuning of the cell back to the normal operating voltage by having valium or valium like substances allow the production of endogenous dopamine from the cell and thereby beginning the process of returning the cell to normal electrical potentials. These changes can only occur if an antagonist molecule has corrected the function of the GABA receptor complex, which was previously damaged by prolonged exposure to endogenous substances with activity at the benzodiazepine receptor site.

This correction is achieved if flumazenil molecules occasionally interact with the receptor sites and help return the cell to normal operating voltages. This corrects the pathological conditions which occur in neurons when the balance between benzodiazepine active molecules and inverse agonists is damaged.

The low infusion rates of flumazenil over a continuous and long term basis, with occasional entry to the cell, corrects the hyperpolarisation voltage levels of these cells, which are in a hyperpolarised state. This invention provides for prolonged infusions for months or years as a means of correcting a large number of diseases which result from damage to cells by injury or other causes.

### Diseases, Disorders and Conditions

The diseases, conditions or disorders that may be treated by the continuous infusion of an antagonist or inverse agonist, such as flumazenil include, but are not limited to: chronic pain, autism, Alzheimer's disease, alcohol addiction, spinal cord injury, multiple sclerosis, chronic fatigue syndrome, acute brain traumatic injury, idiopathic hypersomnia, Parkinson's disease, ischemic brain injury, viral brain injury, epilepsy, Tourette's syndrome, depression, anxiety, schizophrenia, psychosis, Attention Deficit Disorder (ADD), Attention Deficit Hyperactivity Disorder (ADHD), macular degeneration, hearing loss as a result of neural cell damage, tinnitus, diabetes Type I and II which is a result of the malfunction of neural cells, pituitary diseases that do not have releasing factors from the hypothalamus, depression, mania, anxiety, bi-polar. In addition muscle tissue, including cardiac muscle may be damaged in the same way as it is also an electrical tissue similar to that of neurological tissue. The same principal of flumazenil infusions may provide a “screen saver” effect and return muscle tissue to normal function.

Furthermore, cell death in tissues that have significant voltage activity may also be treated by the methods and implants of the present invention. These include many different cell groups, for example, neural and muscle tissue. Most other cells in the body have the ability to also carry some voltage signals and antagonists or inverse agonists, such flumazenil may well be able, through the correction of hyperpolarisation of the cell, to correct other cell functions as well. In conditions such as heart attack, the loss of muscle tissue after an episode of acute ischemia might be reduced by flumazenil infusions.

### Experiments in Sheep

We conducted a series of experiments in sheep to determine the safety of flumazenil infusion, in order to be able to treat the hyperpolarisation and benzodiazepine receptor problem that occurs in a variety of neurological disorders.

Our sheep experiments had proven that sheep exposed for six months to flumazenil at 4 mg/day to 40 mg/day had no illness from the prolonged exposure to constantly infused flumazenil at these rates. This is the only constant infused experiments in animals at these rates to exclude disease. The examination of the sheep brain following the prolonged exposure showed some hippocampal hypertrophy but this would normally be seen as an advantage to the patient.

### Infusion of Flumazenil for Treatment of Benzodiazepine Addiction

In a series of more than 500 patients, infusions of flumazenil were administered using intravenous, subcutaneous delivery and implant delivery systems. The patient numbers included 100 individuals treated with intravenous infusions, more than 300 individuals with subcutaneous delivery of flumazenil and more than 20 individuals with flumazenil delivery from implants.

The delivery rates tested were from 16 microgram per hour up to 330,000 micrograms per hour for 1 day up until 4 days.

Previous studies by other research groups demonstrated that infusion rates of 1mg of benzodiazepine receptor antagonist over three (3) minutes (equivalent to 20,000,000 micrograms per hour) induced panic attacks in patients. It was suggested by the applicant that the benzodiazepine antagonist could not reach the cell and as such, the receptor could not function when covered by such large doses of benzodiazepine receptor antagonists, causing the panic attacks.

The experiments in the 500 human adult volunteers revealed that those who had damaged their benzodiazepine receptors with the use of prolonged high doses of benzodiazepines gained relief and were able to show the effects of benzodiazepines affecting their cells—usually within hours of commencing flumazenil at rates of 1-8 mg of flumazenil per day.

It was observed in patients receiving the flumazenil infusions that there was a marked improvement in the symptoms treated by benzodiazepines. This supported our understanding that the benzodiazepine used to treat patient symptoms was now able to enter the cell and that this improved within hours of starting the infusion and could be maintained for up to 3 to 4 days, or longer.

The applicant concludes that from these experiments, long term flumazenil infusion improves brain function in individuals where benzodiazepine has damaged benzodiazepine receptor sites and that long term flumazenil is safe for use in human beings.

Applicant believes that continual flumazenil infusion is useful to treat disorders, diseases or conditions where excessive GABA tone may have commenced initially with an injury which has resulted from trauma, infection, immune disorders where neurons were damaged or from acute vascular injury.

The applicant suggests that initial injury to the cell causes a rise in endogenous benzodiazepine which causes hyperpolarisation of neural cells following the intake of chloride ions into the cell. This raises the electrical potential of the cell so much that electrical currents in the brain exclude that damaged cell, allowing it to rest and recover. In a short term injury the cell recovers so quickly that damage to the benzodiazepine complex on the GABA receptor does not occur. In this case endogenous benzodiazepine levels settle quickly and so endogenous benzodiazepine continue to reach the cell and allow the release of dopamine by the cell. This dopamine and other functions of the cell allow the cell to correct the hyperpolarisation of the cell so that the cell following injury can return to its normal functioning conducting currents.

From the above experiments we have demonstrated that by constant infusion of flumazenil at rates as low as 0.4 mg/day to rates as high as 8 mg/ day we can return “sick” or damaged hyperpolarised cells to normal function where the electrical currents are conducted as normal.

### Parkinson's Disease

A study was conducted to test the theory that the loss of function of neuronal cells from hyperpolarisation in a patient suffering from Parkinson's disease could be reversed, improved or corrected. An infusion of flumazenil at a rate of 170 micrograms per hour was administered to an adult patient over a period of 7 days. In addition, the patient was also given 10 mg of valium per day to facilitate the “sick cells” in the substantia niagra region of the brain stem, being able to produce their own dopamine again.

The patient was asked to perform the “finger tap test”, which is an indicator of the extent of Parkinson's disease. Briefly, the patient is asked to tap an index finger as fast as possible for one (1) minute (CNS tap test). The greater the number of taps per minute, the greater the improvement.

The following results were observed over four hours of treatment to the patient.

| Time from infusion | Tapping score |
|---|---|
| (hours) | (taps per minute) |
| 0 | 40 |
| 2 | 50 |
| 3 | 60 |
| 4 | 73 |

The study results strongly support that in the first four (4) hours after commencing the flumazenil antagonist treatment to correct cell function, the benzodiazepine receptor sites were able to uptake endogenous and/or exogenous benzodiazepine molecules.

This experiment provides validation that as a result of a low dose of infused flumazenil, the cell was now able to produce its own dopamine. The recovery of receptor function is suggested to be caused by correcting the ability of the cell to respond to benzodiazepine.

Whilst it is known that valium makes the body release endogenous dopamine, it is suggested that patients with Parkinson's disease cannot respond to valium as the high levels of endogenous benzodiazepine have effectively damaged the ability of the receptors to respond to benzodiazepine.

In the case of the experiment with the Parkinson's disease patient discussed above, the effect of valium on the cell causes the release of endogenous dopamine. The evidence for this was that the patient was literally able to reduce his dopamine intake obtained via his medication (L-dopa). That is, he reduced his medication dosage from 1500 mg/day to 250 mg/day within days of commencing infusion treatment. This is a result of the cells now responding to the benzodiazepine, and releasing their own dopamine. We observed in the. days following the infusion that the cells that had not been able to release their own dopamine now had the ability to respond to valium, which was recovered in the same way that we had seen in the response to endogenous benzodiazepine in benzodiazepine addicted experiments.

### Idiopathic Hypersomnia

We tested this model of disease again in two idiopathic hypersomnia patients. Both individuals had severe sleep drunkenness for over 18 months (Patient 1) and for over 13 years (Patient 2). We believe this illness to be due to the damage in the balance between an initial insult that had caused hyperpolarisation of cells which made them nonresponsive to benzodiazepine, as the prolonged presence of high levels of endogenous benzodiazepines continues long after the initial insult or disease. The homeostatic mechanisms that balance the delivery of dopamine to their cells responded to the damaged benzodiazepine receptors by maintaining the endogenous benzodiazepines at a high level in order to get some benzodiazepines to their cells. This inadequate dopamine delivered by response to valium was not occurring when we met these patients and so their cells remained unable to conduct currents and also unable to recover from their hyperpolarisation in the absence of dopamine.

We therefore administered flumazenil at a rate of 4 mg per day to the first of these hypersomnia patients (Patient 1) based on the results and safety demonstrated in our earlier experiments with prolonged infusions to correct cell function. Patient 2 was treated in a similar way.

Within 3 days both patients had an improvement in receptor function that allowed their cells (neurons) to conduct currents and both reported full relief from the sleep drunkenness. We then progressed to flumazenil infusion at 2 mg/day by implant in Patient 1, and within 7 days of this saw a full return to normal health with no sign of cells being hyperpolarised and not functioning.

Our series of experiments demonstrate the pattern of disease with neurons that appear to be dead or non-functioning because of hyperpolarisation that cannot be corrected until the benzodiazepine receptor is returned to normal functioning.

The previous reports of cell death or diminished receptors with the following diseases, such as autism, Alzheimer's disease, spinal cord injury, multiple sclerosis, acute brain traumatic injury, hypersomnia, Parkinson's disease, ischemic brain injury, or vial brain injury, appear to be experimental error. We have demonstrated in at least Parkinson's disease that we can return the neuronal cells to normal function. We have also confirmed in hypersomnia that we can return the neurons to normal function.

### Anxiety, Depression and Psychosis

We observed the response in about 500 patients treated with flumazenil at rates from 20 to 300 microgram per hour with subcutaneous and intravenous infusions.

Within 3 days of commencing infusions (5 days at the lower end of this infusion rates) the patients show a significant improvement in calmness and the ability to relate to others. In most patients we terminated the infusions for cost or convenience reasons and noted a return in anxiety which was less than the time before the infusion but more than at the time of the infusion.

Those with depression, anxiety and fear also showed a confidence in themselves during the infusion treatment. These changes appeared to be less at the 20 mg/hour end of the scale but very marked beyond 40 mg/hour with the top end of the scale extending to. 300 mg/hour and beyond.

More than 20 of the 500 were previously diagnosed with psychosis and these also lifted in their depression and in their anxiety symptoms.

A return to natural sleep was achieved by nearly all of the 500, with the exception of one patient who reported difficulty getting to sleep.

Alertness was also monitored during infusion treatment. During the time of being awake most of the 500 observed an increase in problem solving and reported a decrease in distractibility.

In summary, the implants tested in more than 30 patients alleviated the symptoms of depression, anxiety, sleeplessness, psychosis, and alertness for up to 300 days at a time.

These implants are made with a membrane made of poly-lactic acid which surrounds a mass of poly-lactic acid and flumazenil. The supersaturated solution between the tablet and the membrane allows the rate of dissolving of the multiple of tablets to be controlled by the membrane. The membrane also controls the rate the drug crosses the membrane into the subcutaneous tissues where it is transferred to the blood stream.

This new implant now allows this control of symptoms of anxiety, psychosis, depression, loss of confidence and general well-being to be achieved for up to 300 days. This is literally the first implantable drug to treat depression, anxiety, sleeplessness and psychosis for hundreds of days with a single treatment. With a study base of hundreds of patients these conclusions are quite robust.

While we have adequately demonstrated return to function we are in the process of demonstrating prolonged cell survival as correction of the pathways will protect the cell from prolonged hyperpolarisation which leads to cell death.

### Naltrexone Therapy

Our experiments in approximately 6,000 patients treated with naltrexone (release rate of between 0.5 to 20 mg/day) or 1 to 100 micrograms per hour, with similar release rates of antagonists, has confirmed improvements in multiple sclerosis in our patients and also in crohn's disease which are both inflammatory conditions. The L isomer of naltrexone which blocks opiates receptors also reduces dopamine effects which are so useful in neuronal cells. These experiments showing blocking of dopamine is why the combination of right hand isomer of naltrexone and flumazenil in the dose range described is the ideal treatment to arrest multiple sclerosis.

The conclusion from the long series of experiments conducted by our team is that naltrexone and flumazenil combinations and each one separately will control inflammatory disorders and correct neuronal cell function in the neurological diseases described herein.

In addition, the combination of a low continuous infusion of flumazenil together with naltrexone is useful in treating chronic pain.

The ideal products designed and demonstrated from this research are flumazenil implants, naltrexone implants, combinations of both and combinations that include both where the right hand isomer of naltrexone is used to decrease the inflammatory cascade with no decrease in opiate or dopamine function. These are the products that save neuronal cells from diseases of the nervous system.

### Implantable Dosage Form of Flumazenil

Applicant has surprisingly found that flumazenil can be administered in an implantable dosage formulation to treat neurological disorders and possibly other diseases or disorders associated with damage to cells having an electrical potential, as listed herein. The implant is designed to release a constant and steady amount of flumazenil over an extended period of time.

It should be appreciated that the implants of this invention may be administered to a patient in any suitable manner for which the active agent(s) is designed to be administered. Most preferably, the preparations are designed for subcutaneous administration, preferably in the abdominal wall.

The implants of the present invention are adapted to deliver active agent at a constant rate for an extended period of time. Where the active agent is, for example, flumazenil the rate of delivery is preferably about 10 to 300 micrograms per hour. The release rate may also be between 300 to 500 micrograms per hour, or 500 to 1000 micrograms per hour. The release rate may be up to 20,000 micrograms per hour.

In addition, the implantable dosage form of flumazenil may achieve a constant rate of delivery of flumazenil from 100 days to 300 days, or longer.

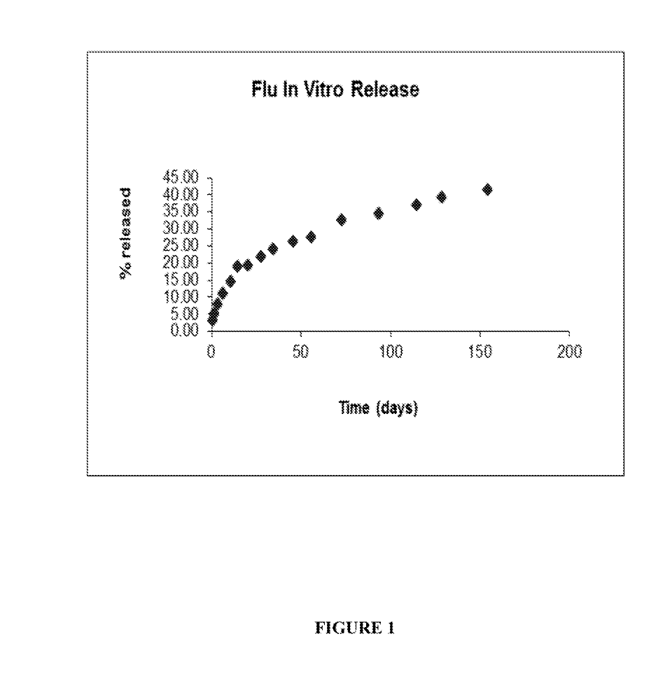

Flumazenil has been formulated into an implant dosage form. The average weight of the tablet was 253 mg, containing 99.48 mg of flumazenil. The tablet was coated with 20% PLA solution of release testing. The solubility of flumazenil at pH 1.2 is 3 mg/ml, and at a pH of 7.5 has a lower solubility of only 0.6 mg/l. FIG. 1 shows implants designed with a rate limiting membrane around tablets that contain a super-saturated solution allowing the gradual release of flumazenil and limiting the rate of dissolving of the tablet.

We have designed an implant that permits the continual release of the active agent from the implant. The active agent is surrounded by one or more membrane layers. A supersaturated solution of active agent is formed between the mass of active agent and the membrane. The implant is thus able to release the active agent(s) at a controlled release rate.

The polymeric matrix material of the implants of the present invention is a biocompatible and biodegradable polymeric material. Preferably, the biodegradable polymer used in the preparation of the pharmaceutical preparation is long lasting. The matrix material should be biodegradable in the sense that the polymeric material should degrade by bodily processes to products readily disposable by the body and should not accumulate in the body. Preferably, the length of time that the biodegradable polymer stays intact is more than 20 days, more preferably the biodegradable polymer stays intact for a length of time of over 45 days.

More preferably the biodegradable polymer stays intact for a length time of over 50 days. More preferably the biodegradable polymer stays intact for a length of time of over 3 months, still more preferably, more than 6 months and still more preferably more than 1 year.

The coating should however allow the active agent to diffuse out of the implant and into the surrounding blood stream and its thickness can be altered to control this role. Therefore when the coating is present, the implant is still able to release active agent. Suitable examples of polymeric matrix materials include poly(glycolic acid), poly-D,L-lactic acid, poly-L-lactic acid, copolymers of the foregoing, poly(aliphatic carboxylic acids), copolyoxaates, polycaprolactone, polydioxonone, poly(ortho carbonates), poly(acetals), poly(lactic acid caprolactone), polyorthoesters, poly(glycolic acid-caprolactone), polyanhydrides, and natural polymers including albumin, casein, and waxes, such as, glycerol mono-and distearate, and the like. The preferred polymer for use in the practice of this invention is Pura-DL-lactide with a molecular weight of 24 800 and inherent viscosity of 0.53 dl/g. An example of another biodegradable polymer is Poly-DL-lactide/glycolide copolymer with inherent viscosity of 0.9 dl/g.

The inventors have also found that coating the compressed tablets formed from a plurality of microcapsules with one or more layers of a biodegradable polymer reduces the absorption rate of the active ingredient. Another effect of using a coating around the tablets is to reduce the risk of tissue irritation caused by direct contact of the active agent with surrounding tissue. Preferably, a tablet is coated with at least one layer of biodegradable polymer. It is more preferable if a tablet is coated with at least 2 layers of biodegradable polymer. It is even more preferable if a tablet is coated with at least 3 layers or more of biodegradable polymer.

A plurality of tablets can be coated with a biodegradable polymer to further reduce the absorption rate of the active agent. Thus two or more tablets can be formed into one pellet by coating the tablets with a biodegradable polymer. The rate of absorption of such a pellet is lower than that of an equivalent sized implant made from one tablet. This may be due to the number of coatings of biodegradable polymer and reduced surface area of active agent exposed. Preferably there is one coating of biodegradable polymer. More preferably, there are two coatings of biodegradable polymer. Still more preferably, there are three coatings of biodegradable polymer.

The pellets described above can contain one or more tablets comprising different active agents, each active agent having different rates of release. For example one tablet may comprise of flumazenil, a second tablet may comprise naltrexone.

The thickness of the coating of biodegradable polymer surrounding the tablet may affect the absorption rate of the active ingredient. The greater the thickness of the coating, the greater the reduction in absorption rate of the active agent. Preferably the thickness of the coating is 0.1 mm to 1 mm. More preferably the thickness is 0.3 mm to 0.7 mm. Still more preferably the thickness is 0.4 mm to 0.6 mm. An example of a suitable thickness of the coating is 0.6 mm.

The lipophicity of the active agent may also affect the rate of release through the polymer layer, i.e. a more lipophilic active agent will require a thicker polymer layer

The microcapsule product used in the present invention can be prepared by any method capable of producing microcapsules in a size range acceptable for use in the compressed tablets. In these methods, the material to be encapsulated (ie the active agents) is generally dissolved, dispersed, or emulsified, using known mixing techniques, in a solvent containing the wall-forming material. Solvent is then removed from the microcapsules and thereafter the microcapsule product is obtained. An example of a conventional microencapsulation process is disclosed in U.S. Pat. No. 3,737,337 wherein a solution of a wall or shell forming polymeric material in a solvent is prepared. The solvent is only partially miscible in water. A solid or core material is dissolved or dispersed in the polymer-containing solution and, thereafter, the core-material-containing solution is dispersed in an aqueous liquid that is immiscible in the organic solvent in order to remove solvent from the microcapsules. Another example of a process in which solvent is removed from microcapsules containing a substance is disclosed in U.S. Pat. No. 3,523,906. In this process, a material to be encapsulated is emulsified in a solution of a polymeric material in a solvent that is immiscible in water and then the emulsion is emulsified in an aqueous solution containing a hydrophilic colloid. Solvent removal from the microcapsules is then accomplished by evaporation and the product is obtained.

The microcapsules can be mixed by size or by type so as to provide for the delivery of active agent to the patient in a multiphasic manner and/or in a manner that provides different agents to the patient at different times, or a mixture of agents at the same time.

Pharmaceutical excipient can also be used in the implants of the invention. Suitable excipient are well known in the art and include starch, cellulose, talc, glucose, lactose, sucrose, gelatin, malt, rice, flour, chalk, silica gel, magnesium stearate, sodium stearate, glycerol monostearate, sodium chloride and dried skim milk.

While pharmaceutical excipients may be incorporated in the inventive pharmaceutical preparation it should be observed that it is not desirable to incorporate significant amounts of disintegration agents that might have the effect of increasing the rate of degradation of the tablet. Such agents are preferably not included in the tablet formulation.

Pharmaceutical preparations prepared according to the invention will have broad application in treating patients in need of long term treatment of the active agents described herein. According to one embodiment the present invention provides a method of treating a patient by administering a pharmaceutical preparation as described herein to the patient. Most preferably the preparation is administered subcutaneously.

In one embodiment, the implant is administered by a method of inserting one or more pharmaceutical preparations of the present invention into a tissue of a patient comprises the following steps:
a) making a single small incision into the tissue with a needle and first sheath;
b) withdrawing the needle from the first sheath, but leaving the first sheath in the tissue;
c) dilating the opening of the incision by inserting a dilator and second sheath of larger diameter through the bore of the first sheath;
d) withdrawing the dilator from the second sheath; and
e) dispensing the preparation by inserting it through the second sheath in the tissue.

If necessary, further dilation of the opening of the initial incision may be achieved by repeating the process of inserting dilators and sheaths of increasing diameter through the bore of sheaths of smaller diameter, until the diameter of the opening of the incision is sufficiently large to receive the preparation. Methods of inserting implants may be found in patent application WO 2002/017971.

The methods and implants described by the present invention are capable of treating a number of different disorders, diseases or conditions, including idiopathic hypersomnia, Parkinson's disease and epilepsy.

### EXAMPLES

### Treatment of Idiopathic Hypersomnia

Idiopathic hypersomnia (IH) is a rare sleep disorder characterized by excessive daytime sleepiness, undisturbed nocturnal sleep, un-refreshing naps, and sleep drunkenness. The rare nature of the disorder has made the conduct of rigorous clinical trials difficult and thus very little is known about the condition. For this reason the treatment of IH has largely focused on the alleviating the symptoms, with the use of stimulant or awake promoting drugs such as modafinil, dextramphetamines and methylphenidates.

Recently, examination of the cerebrospinal fluid (CSF), found that CSF of hyper-somnolent subjects contained a substance that stimulates the function of gamma-aminobutyric acid (GABA) receptors in the presence of GABA in vitro, which was not identified in health controls (Rye et al 2012). The use of substances that antagonize the GABA receptors, such as flumazenil and clarithromycin has been postulated by others as a mechanism for treating idiopathic hypersomnia. Flumazenil is available in an intravenous dosage form. However, large quantities of flumazenil, up to 30 to 60 mg per day are required. Furthermore, the doses must be administered on a regular basis, such as every three hours due to the short half-life of flumazenil.

Another research group has used a single dose injection of flumazenil only. One patient has used long intervals between sublingual doses with weeks after each dose and then hours between that and the next dose but despite years of this treatment, it has not proved practical to other patients with the variation in dosage levels of flumazenil being difficult for most patients. As an alternative, the use of a continuous intravenous infusion of flumazenil was proposed by the inventor.

The first patient (P1) was treated with a 4 day subcutaneous infusion of flumazenil at a rate of 4 mg of flumazenil per day. This is in contrast with an existing patient treated by the Emery Research Group, who indicated a dose of approximately 2 mg per body mass unit per day would be required. For P1, with a BMI of 34 this would equate to 68 mg per day, 17 times what he was given during the infusion. If efficacious, the benefits of using a lower dose would most likely be a reduction in adverse events—most notably the risk of seizure.

During the infusion, the patient reported not having experienced any sleep drunkenness, excessive day time sleepiness, heart palpitations or cold hands and feet (all experienced prior to treatment and common symptoms of IH). The patient P1 also noted a marked improvement in his concentration, memory and attention span. At the time of treatment, the patient scored a 6 out of a possible 8 on the Stanford Sleepiness Scale (SSS) and scored 21 of a possible 24 on the Epworth Sleepiness Scale (ESS). At the completion of the flumazenil treatment, he scored 3 on the SSS and 4 for the ESS.

During the infusion and the two weeks prior, P1 kept a record of his sleepiness and mood, upon awaking, mid-awake and pre-sleep on a scale of 0-10. For sleepiness 0 represented an unavoidable need to sleep, while 10 is feeling awake and rested. Similarly for mood, 0 represents rock bottom, while 10 was chirpy, happy and cheerful. Following treatment both mood and awake-ness improved significantly.

| | Pre treatment | Post treatment |
|---|---|---|
| Sleepiness | | |
| Awaking sleepiness | 1.6 ± 0.8 | 6.2 ± 0.8 |
| Mid-awake sleepiness | 2.2 ± 0.7 | 6.8 ± 0.5 |
| Pre-sleep sleepiness | 1.4 ± 1.0 | 5.8 ± 1.7 |
| Mood | | |
| Awaking mood | 3.0 ± 1.0 | 8.8 ± 0.4 |
| Mid-awake mood | 2.8 ± 1.1 | 8.8 ± 0.5 |
| Pre-sleep mood | 2.6 ± 1.2 | 8.5 ± 0.6 |

At the end of the 4 day period, P1 in discussion with medical staff opted to be treated with a subcutaneous flumazenil implant (5 tablets). He has since been doing very well, with minimal day time sleepiness and no napping,

We have since treated a second, IH patient and the results have been more conservative than noted with P1. The second patient (P2) has noted an increase in mental clarity and his wife has noted that he has been a lot easier to talk to. However, P2 has still been very tired and napping through the day. On the following Saturday his dose of flumazenil was doubled to 8 mg/day we expect to see continued improvement.

### Treatment of Parkinson's Disease

Thus far the use of flumazenil has been examined in two published papers (Ondo & Hunter, 2003; Ondo et al, 2006), both of which give some background as to the reasoning behind its use. Both papers however only used a single bolus dose of flumazenil.

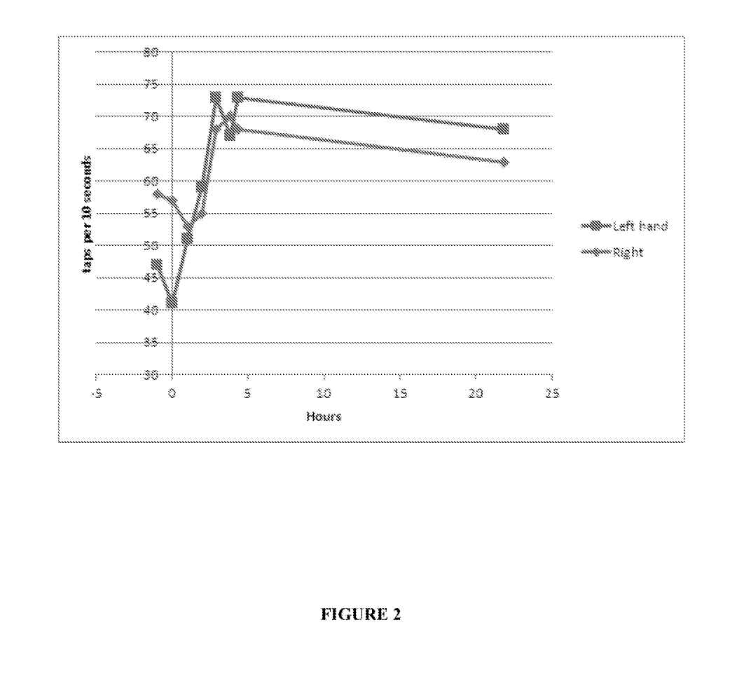

The first patient with Parkinson's Disease was treated on 8 May 2013. We conducted a finger tap test on the patient as an indicator of the disorder. Briefly, the patient is asked to tap his index finger as fast as possible for 10 seconds (CNS tap test). The patient was tested twice before the flumazenil infusion was commenced, and then at approximately 1, 2, 3, 4, 4.5 and 22 hours following the infusion. The results are below are set out in FIG. 2.

Initially the patient had a marked difference between his left and his right hand, consistent with his reports that his left side was more affected than his right (he is left handed). The patient's left hand increased in ability at the first hour and began to perform slightly better than his right hand (which would be expected in a left handed patient).

On 10 May 2013, the patient returned to the treatment clinic, during the previous 2 days he had dramatically reduced his use of his normal Parkinson's medication by at least half.

To boost the efficacy of the treatment we prescribed a benzodiazepine to assist with the cells production of dopamine with the aim of reducing the requirement for medication that provides dopamine. The combination of flumazenil and benzodiazepines has been shown to enhance the release of dopamine in rats (Motzo et al 1997).

### Treatment of Epilepsy

A 22 year patient had been treated by all the epilepsy experts for many years. She is on a disability pension as her epileptic events on maximum treatment still left her with episodes of peti mal (staring spells) when resting at home at least 4-5 times each day. Her most serious problem was light activating her peti mal episodes. This had for many years prevented her going outside her dimly lit house.

The day following her treatment with flumazenil at 170 micrograms per hour she was able to enjoy no fits (none have occurred since (48 hour) and she was for the first time able to go for a walk in the sun for an hour with no hat or sunglasses. This remarkable recovery occurred with no change or interaction with her existing treatment.

### Treatment of Alcohol Addition

We noted in three patients treated with flumazenil implants releasing an estimated 2 mg per day that the patients had no reward and no desire to consume alcohol. This profound reduction in alcohol craving with flumazenil implants has not been described previously and was extremely surprising in casual alcohol abusers.

This observation was unexpected and we have observed this before. It provides support that constant delivery of flumazenil for prolonged periods inhibits or prevents alcohol cravings, use and enjoyment in casual abusers. The effect was described by our patient as one that increased in effect the longer the drug delivery from the implant continued.

1. A method for treating a neurological disease, or other disease or disorder associated with cells having an electrical potential, comprising administering a continuous infusion of a benzodiazepine antagonist or inverse antagonist at a rate of 0.001 micrograms to 20,000 micrograms per hour to a patient in need thereof.

2. The method according to claim 1 wherein the continuous infusion of the benzodiazepine antagonist is maintained for a period of time of more than 4 days, at least 10 days, at least 20 days, at least 30 days, at least 40 days, at least 50 days, at least 60 days, at least 70 days, at least 80 days, at least 90 days, at least 100 days, at least 150 days, at least 200 days, at least 250 days, at least 300 days or more.

3. The method according to claim 1 wherein the continuous infusion of the benzodiazepine antagonist is maintained indefinitely.

4. An implant comprising a benzodiazepine antagonist, wherein the implant comprises at least a benzodiazepine antagonist or inverse antagonist in an amount sufficient for the implant to release the benzodiazepine antagonist at a continuous rate of 0.001 micrograms to 20,000 micrograms per hour for a prolonged period of time.

5. The implant according to claim 4 wherein the antagonist is flumazenil.

6. The implant according to claim 4 or 5 wherein the benzodiazepine antagonist is released at a rate of between 300 to 500 micrograms per hour.

7. The implant according to claim 4 or 5 wherein the benzodiazepine antagonist is released at between 500 to 1,000 micrograms per hour.

8. The implant according to claim 4 or 5 wherein the benzodiazepine antagonist is released at a rate of up to 20,000 micrograms per hour.

9. The implant according to any one of claims 4 to 8 wherein the prolonged period of time is more than 4 days, at least 10 days, at least 20 days, at least 30 days, at least 40 days, at least 50 days, at least 60 days, at least 70 days, at least 80 days, at least 90 days, at least 100 days, at least 150 days, at least 200 days, at least 250 days, at least 300 days or more.

10. Use of an implant to treat a neurological disease, or other disease or disorder associated with cells having an electrical potential in a patient, wherein the implant comprises a benzodiazepine antagonist, wherein the implant comprises at least a benzodiazepine antagonist or inverse antagonist in an amount sufficient for the implant to release the benzodiazepine antagonist at a continuous rate of 0.001 micrograms to 20,000 micrograms per hour for a prolonged period of time.

11. The use of an implant according to claim 10 wherein the prolonged period of time is for the life of the patient.

12. A method for treating a neurological disease, or other disease or disorder associated with cells having an electrical potential, comprising administering a continuous infusion of flumazenil together with a second benzodiazepine antagonist or inverse agonist.

13. The method according to claim 12 wherein the second benzodiazepine antagonist or inverse agonist is valium or a valium-like substance.

14. An implant comprising flumazenil and the L-isomer of naltrexone for the treatment of a neurological disorder, disease or condition.

15. An implant comprising flumazenil and the L-isomer of naltrexone for the treatment of a neurological disorder, disease or condition.

16. The method according to any one of claim 1-3 or 12-13, or the implant according to any one of claim 4-9 or 14-15, or the use according to claim 10 or 11, wherein the diseases, conditions or disorders that may be treated by the continuous infusion of an antagonist or inverse agonist, such as flumazenil include, but are not limited to: chronic pain, autism, Alzheimer's disease, alcohol addition, spinal cord injury, multiple sclerosis, chronic fatigue syndrome, acute brain traumatic injury, idiopathic hypersomnia, Parkinson's disease, ischemic brain injury, viral brain injury, epilepsy, Tourette's syndrome, depression, anxiety, schizophrenia, psychosis, Attention Deficit Disorder (ADD), Attention Deficit Hyperactivity Disorder (ADHD), macular degeneration, hearing loss as a result of neural cell damage, tinnitus, diabetes Type I and II which is a result of the malfunction of neural cells, pituitary diseases that do not have releasing factors from the hypothalamus, depression, mania, anxiety, or bi-polar disorder.
